(19) United States
(12) Patent Application Publication
(10) Pub. No.: US 2020/0345539 A1
(43) Pub. Date: Nov. 5, 2020

(54) Multifunctional Smart Cushion

(71) Applicant: Jiangxi Lanzhen Intelligent Technology Co., Ltd., Nanchang (CN)

(72) Inventor: Jianping MAO, Nanchang (CN)

(73) Assignee: Jiangxi Lanzhen Intelligent Technology Co., Ltd., Nanchang (CN)

(21) Appl. No.: 16/451,033

(22) Filed: Jun. 25, 2019

(30) Foreign Application Priority Data
Apr. 30, 2019 (CN) 201910364908.3

(51) Int. Cl. A61F 7/02; H04M 1/725; A61H 7/00; A61M 21/00

(52) U.S. Cl. CPC A61F 7/02; H04M 1/7253; G10L 15/22; A61H 7/00; A61M 21/00; H04M 1/72533

(57) ABSTRACT

A multifunctional smart cushion includes a smart cushion main body and a mobile terminal. The smart cushion main body includes a smart cushion cover, a structural portion, and a core portion. The structural portion is located around the core portion. The smart cushion cover covers the structural portion and the core portion. The structural portion is provided with a power supply and a controller. The core portion is provided with a fan, a heater, a temperature sensor, a reflux guiding channel, a therapy device, and a massage device. The fan, heater, temperature sensor, therapy device, and massage device are all connected to the power supply through the controller. The mobile terminal is connected to the controller via a wireless communication.

### CROSS REFERENCE TO RELATED APPLICATIONS

This application is based upon and claims priority to Chinese Patent Application No. 201910364908.3, filed on Apr. 30, 2019 the entire contents of which are incorporated herein by reference.

### TECHNICAL FIELD

The present invention belongs to the technical field of smart cushions, and particularly relates to a multifunctional smart cushion.

### BACKGROUND

With the continuous development of society, a variety of smart cushions have been developed to meet different needs of people's work and household life. In general, the smart cushion consisting of a base and a backrest, that are made of soft cushions, has good ventilation and heating effects.

At present, the smart cushions popular in the market work according to the following principles. Using the AC power supply, the smart cushion is heated by an enclosed heater in which the heating wire is enclosed. The heat of the human body is dissipated and blown out by a blower, so as to realize the ventilation of the smart cushion. Further, the desired temperature level is selected by manually controlling the modes.

However, on the downside, the existing smart cushion has a single function and single control and operation mode, and there are limitations on the use. The use of the blower cannot timely dissipate the heat from the parts blocked by the human body. As a result, the heat may accumulate locally due to the poor heat circulation in the enclosed heater, thereby affecting the user's experience. Moreover, the temperature modes have default settings, so the temperature cannot be precisely controlled, and the user cannot set the operation of the smart cushion independently as per need.

### SUMMARY

In order to eliminate the technical defects and drawbacks of the prior art, the present invention provides a multifunctional smart cushion. To solve the technical problems, the technical solutions of the present invention are as follows.

A multifunctional smart cushion includes a smart cushion main body and a mobile terminal. The smart cushion main body includes a smart cushion cover, a structural portion, and a core portion. The structural portion is located around the core portion, and the smart cushion cover covers the core portion and the structural portion.

The structural portion is provided with a power supply and a controller.

The core portion is provided with a fan, a heater, a temperature sensor, a reflux guiding channel, a therapy device, and a massage device.

The fan, the heater, the temperature sensor, the therapy device, and the massage device are all connected to the power supply through the controller.

The mobile terminal is connected to the controller via a wireless communication.

In one embodiment of the present invention, the power supply is a mobile power supply, and the voltage of the mobile power supply does not exceed a safe voltage level for the human body.

In one embodiment of the present invention, the controller includes:

a processor configured to receive an operation signal and send a control signal;

a switch button configured to switch between different working modes;

a Bluetooth unit configured to receive a Bluetooth command sent by a user and convert the Bluetooth command into a Bluetooth operation signal;

a voice recognizing unit configured to receive a voice command issued by the user and convert the voice command into a voice operation signal; and

a touch control unit configured to receive a touch control command created by the user and convert the touch control command into a touch control operation signal.

Moreover, the Bluetooth unit, the voice recognizing unit, and the touch control unit are all connected to the processor through the switch button.

In one embodiment of the present invention, the Bluetooth command includes a Bluetooth operation command or a Bluetooth voice command.

In one embodiment of the present invention, the controller further includes:

an aroma diffusing unit connected to the processor to act in response to an aroma diffusing unit control signal sent by the processor.

In one embodiment of the present invention, the fan is a scroll-type exhaust fan.

In one embodiment of the present invention, the heater includes at least two layers of breathable materials and a heating wire arranged between two adjacent layers of the breathable materials.

In one embodiment of the present invention, the reflux guiding channel includes at least two ventilation pipes perpendicular to each other; and

the fan is fixedly provided at an intersection of the two ventilation pipes.

In one embodiment of the present invention, a vacuum fixation installation system is further provided at a bottom of the smart cushion main body.

From here we see that by configuring the multifunctional smart cushion of the present invention with a scroll-type exhaust fan, the heat in the smart cushion is dissipated by air extraction, which avoids the problem of difficulty in timely discharging the heat from the parts blocked by the human body by using the traditional blowing method. The air flow can be drawn out of the smart cushion in time through the reflux guiding channel, so the problems of heat reflux and noise are solved. By providing the heater made of breathable material, the heat circulation inside the smart cushion is improved, so the problem of local heat accumulation caused by the poor heat circulation of the traditional heater can be avoided. The vacuum fixation installation system enables a suction fixation, which is attractive and less likely to cause damage to the smart cushion. In addition, the multifunctional smart cushion of the present invention is versatile and has a wide application range as it integrates the functions of aroma diffusion, therapy, and massage.

Other aspects and features of the present invention will become clearer through below detailed description with reference to the drawings. However, it should be understood that the drawings are merely designed for the interpretation purposes and are not intended to limit the scope of the present invention, because the scope of the present invention shall be referred to the appended claims. It should also be understood that, unless otherwise indicated, the drawings are not necessarily drawn in proportion, they merely intended to conceptually illustrate the structures and processes described herein.

### BRIEF DESCRIPTION OF THE DRAWINGS

FIG. 1 is a top view schematic diagram showing the structure of a multifunctional smart cushion according to an embodiment of the present application;

FIG. 2 is a principle diagram of a multifunction smart cushion in control of a Bluetooth unit according to an embodiment of the present application;

FIG. 3 is a principle diagram of a multifunction smart cushion in control of a voice recognizing unit according to an embodiment of the present application;

FIG. 4 is a control principle diagram of a multifunction smart cushion in control of a touch control unit according to an embodiment of the present application;

FIG. 5 is a control principle schematic diagram of a multifunction smart cushion when a controller is used as an aroma diffusing unit according to an embodiment of the present application;

FIG. 6 is a control principle diagram of a multifunction smart cushion which is used as a therapy cushion according to an embodiment of the present application;

FIG. 7 is a control principle diagram of a multifunction smart cushion which is used as a massage cushion according to an embodiment of the present application; and

FIG. 8 is a side view schematic diagram showing the structure of a reflux guiding channel of a multifunctional smart cushion according to an embodiment of the present application.

### DETAILED DESCRIPTION OF THE EMBODIMENTS

To clarify the above objectives, features, and advantages of the present invention, the specific embodiments of the present invention will be described in detail hereinafter with reference to the drawings.

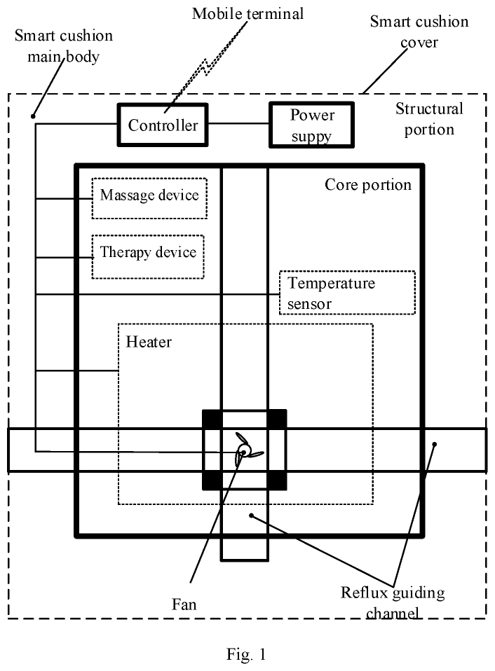

Referring to FIG. 1, FIG. 1 shows a top view schematic diagram showing the structure of a multifunctional smart cushion according to an embodiment of the present application. This embodiment provides a multifunctional smart cushion, including: a smart cushion main body and a mobile terminal. The smart cushion main body includes a smart cushion cover, a structural portion, and a core portion. The structural portion is located around the core portion, and the smart cushion cover covers over the core portion and the structural portion.

The structural portion is provided with a power supply and a controller.

The core portion is provided with a fan, a heater, a temperature sensor, a reflux guiding channel, a therapy device, and a massage device.

The fan, the heater, the temperature sensor, the therapy device, and the massage device are all connected to the power supply through the controller.

The mobile terminal is connected to the controller via a wireless communication.

Specifically, the existing smart cushion is powered by the AC power supply and uses the enclosed heater where the heating wire is enclosed to supply heat to the smart cushion. Also, the blower is used to dissipate the excess heat out of the smart cushion by blowing, so as to achieve the ventilation, and the temperature level is selected by manually controlling the modes. Such a smart cushion has the following problems. The smart cushion has a single function and limitations on the use. The use of the blower can cause the case where the heat cannot be timely dissipated from the parts blocked by the human body. As a result, the heat will accumulate locally due to the poor heat circulation in the enclosed heater, thereby affecting the user's experience. Moreover, the temperature modes have default settings, so the temperature cannot be precisely controlled, and the users cannot set the operation of the smart cushion independently as per need.

In order to solve the above problems, this embodiment of the present application provides a multifunctional smart cushion, according to which a temperature sensor is provided to monitor the temperature information of the smart cushion in real time, and the temperature information is sent to the mobile terminal as a feedback to the user through the Bluetooth unit of the controller or through the built-in voice recognizing unit or touch control unit of the controller. The smart cushion is regulated by a user by sending a control command to the mobile terminal or the built-in voice recognizing unit and the touch control unit of the controller. The user sends a specific control command to the controller, and the controller, in response, sends a control signal according to the specific control command to adjust the working mode of the heater or the fan to achieve precise regulation and control of the temperature. In addition, the smart cushion of the present application can also receive an aroma diffusing command, a therapy command, or a massage command from the user through the controller to shift the role of the smart cushion to a therapy cushion or a massage cushion according to the demands, and the controller can be used as an aroma diffusing unit independently.

In a specific embodiment of the present invention, the power supply is a mobile power supply, and the voltage of the mobile power supply does not exceed the safe voltage level for the human body.

Specifically, the use of the mobile power supply having its voltage not exceeding the safe voltage level for the human body has the advantages of being safe, handy, and has small size. Moreover, the mobile power supply can automatically recognize the product connected to it and supply power. When the mobile power supply is disconnected from the product, the mobile power supply can automatically recognize the situation and stop supplying power. In this way, greater convenience is offered to the users, as they do not need to turn on or turn off the power supply function of the mobile power supply every time they connect or disconnect the product. Therefore, it is more convenient and more user-friendly during use, and can also save more power of the mobile power supply. With these characteristics, the multifunctional smart cushion of the present invention can be used in various occasions such as office, home, car, train, airplane, cinema, waiting room, outdoor, field, travel, restaurant, etc. Users can regulate and select specific functions according to their needs.

In a specific embodiment of the present invention, the controller includes:

a processor, configured to receive an operation signal, and send a control signal;

a switch button, configured to switch between different working modes;

a Bluetooth unit, configured to receive a Bluetooth command sent by a user, and convert the Bluetooth command into a Bluetooth operation signal;

a voice recognizing unit, configured to receive a voice command issued by the user, and convert the voice command into a voice operation signal; and

a touch control unit, configured to receive a touch control command created by the user, and convert the touch control command into a touch control operation signal.

Specifically, the Bluetooth unit, the voice recognizing unit, and the touch control unit are all connected to the processor through the switch button.

In a specific embodiment of the present invention, the Bluetooth command includes a Bluetooth operation command or a Bluetooth voice command.

Specifically, the controller is provided with a fingerprint switch button, and the controller can be turned on or off by fingerprint identification during use. The temperature sensor is configured to monitor the temperature inside the smart cushion in real time and give feedback to the processor in real time based on the temperature information. The processor sends the temperature information to the user in different ways. The user can switch among different control modes by operating the switch button.

In addition, according to the smart cushion of the embodiment of the present application, the core portion is further provided with a pressure sensing device. The pressure sensing device is connected to the controller for sensing the pressure or gravity signal of the human body to regulate the startup and shutdown of the controller. The specific operating principle is: when the pressure sensing device in the smart cushion senses the pressure or gravity of the human body, a pressure signal is generated and transmitted to the controller, and the controller recognizes the pressure signal and starts up; and when the human body leaves the smart cushion, no pressure or gravity of the human body is applied to the pressure sensing device in the smart cushion, so the pressure signal disappears, and the controller is shut down.

As shown in FIG. 2, when the smart cushion is switched to the Bluetooth unit, the voice recognizing unit and the touch control unit stop working, the user turns on the mobile terminal, and the processor transmits the temperature information to the mobile terminal via Bluetooth for the user's reference and receives the Bluetooth command issued by the user through the mobile terminal. The Bluetooth command described here may be a specific operation of the user on the mobile terminal, namely a Bluetooth operation command, or may be a Bluetooth voice command created by the mobile terminal according to user's voice input operation. The Bluetooth unit receives the Bluetooth command issued by the user, converts the Bluetooth command into a Bluetooth operation signal, and sends the Bluetooth operation signal to the processor. After receiving the Bluetooth operation signal, the processor regulates the smart cushion by adjusting the working mode of the heater or the fan. The temperature sensor records the change of the temperature information in real time and gives feedback to the processor in real time based on the temperature change information. Then, the processor transmits the temperature information to the mobile terminal through a Bluetooth feedback to update the temperature information and other information for the user's reference.

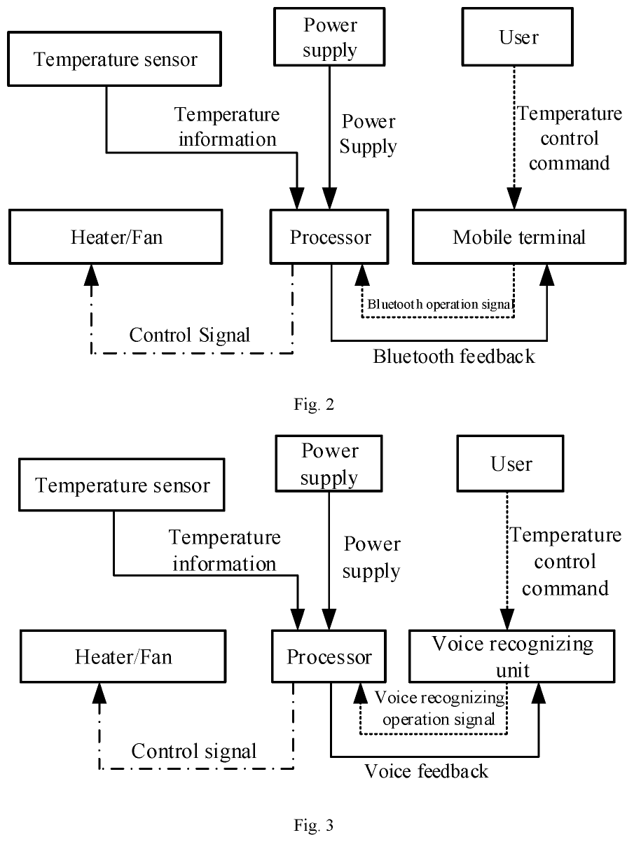

As shown in FIG. 3, when the smart cushion is switched to the voice recognizing unit, the Bluetooth unit and the touch control unit stop working, and the processor transmits the temperature information to the user by voice broadcast and receives the voice command issued by the user. The voice recognizing unit receives the voice command issued by the user, converts the voice command into a voice operation signal, and sends the voice command signal to the processor. After receiving the voice operation signal, the processor regulates the smart cushion by adjusting the working mode of the heater or the fan. The temperature sensor records the change of the temperature information in real time and gives feedback to the processor in real time based on the temperature change information. Then, the processor transmits the temperature information to the user through a voice feedback to update the temperature information and other information for the user's reference.

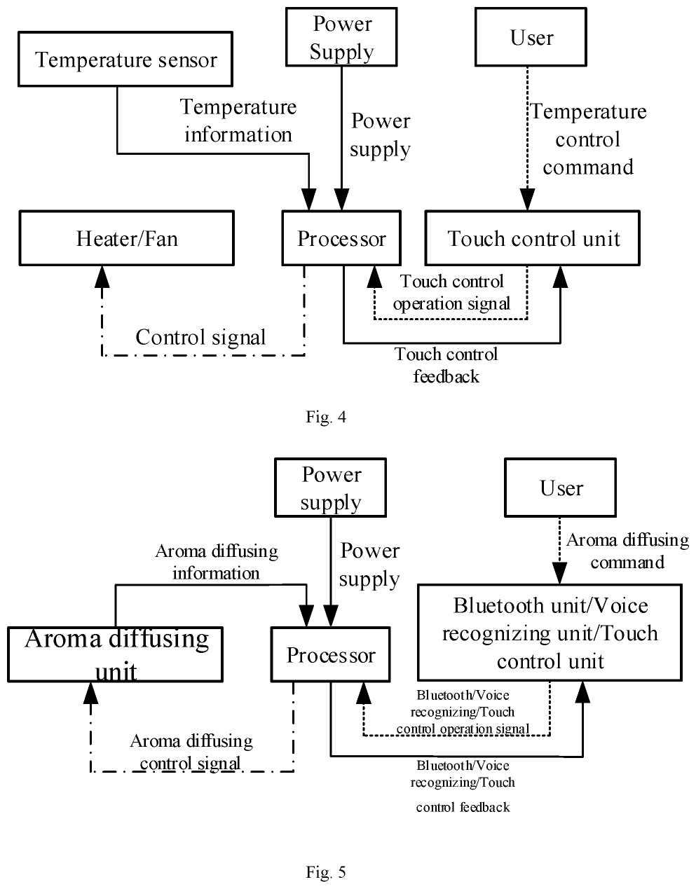

As shown in FIG. 4, when the smart cushion is switched to the touch control unit, the Bluetooth unit and the voice recognizing unit stop working, the processor transmits the temperature information to the user for reference through the touch screen control panel, and the user sends a touch control command by operating the touch screen control panel. The touch control unit receives the touch control command issued by the user, converts the touch control command into a touch control operation signal, and sends the touch control operation signal to the processor. After receiving the touch control operation signal, the processor regulates the smart cushion by adjusting the working mode of the heater or the fan. The temperature sensor records the change of the temperature information in real time and gives feedback to the processor in real time based on the temperature change information. Then, the processor transmits the temperature information to the user through a touch control feedback to update the temperature information and other information for the user's reference.

In a specific embodiment of the present invention, the controller further includes:

an aroma diffusing unit, connected to the processor, to act in response to an aroma diffusing control signal sent by the processor.

Specifically, as shown in FIG. 5, in the embodiment of the present application, the aroma diffusing command issued by the user can be received by the Bluetooth unit, the voice recognizing unit or the touch control unit to control the processor to regulate the operation of the aroma diffusing unit. The Bluetooth command from the user can be received by the Bluetooth unit, the touch control command from the user can be received by the touch control unit, or the voice command from the user can be received by the voice recognizing unit, and the aroma diffusing command is converted into an aroma diffusing operation signal to be transmitted to the processor. It should be noted that here the processor is only allowed to receive one type of aroma diffusing operation signal at one time. The processor receives the aroma diffusing operation signal and sends the aroma diffusing control signal to control the operation of the aroma diffusing unit. The controller can be used as an independent aroma diffuser product. In specific use, the Bluetooth unit, the voice recognizing unit, or the touch control unit receives the aroma diffusing command issued by the user and converts the aroma diffusing command into an aroma diffusing operation signal. The processor receives the aroma diffusing operation signal and sends an aroma diffusing control signal to the aroma diffusing unit. The aroma diffusing unit acts in response to the aroma diffusing control signal received and gives feedback to the user in real time based on the working status of the aroma diffuser.

In addition, in the embodiment of the present application, the therapy command issued by the user can be received by the Bluetooth unit, the voice recognizing unit, or the touch control unit to control the processor to regulate the operation of the therapy unit. In this case, the smart cushion is used as a therapy cushion. According to the traditional therapy, under a specific context and in a purpose-built wooden room, a pile of special rock is heated on a stove to reach a temperature of 70° C. or above, and then a small amount of water is sprinkled on the rock to generate powerful steam, so as to improve blood circulation and completely relax the muscles of all parts of the body, thereby achieving the purpose of eliminating fatigue, restoring physical strength, and refreshing user's spirit. With its inherent health care effects, traditional therapy provides superior enjoyment and leisure for tired people. However, as it requires a specific context and high cost, people cannot use this kind of therapy anytime and anywhere. With these limitations on the use range, traditional therapy is far from meeting people's real needs and requirements. Moreover, since the traditional therapy only has a simple and single therapeutic effect, it has no longer been able to meet the actual needs of users. The users have a few choices on the temperature of the traditional therapy because the therapy temperature is pre-set, so they have no access to change the temperature as per need, and the personalized needs and experience of the users cannot be satisfied. Moreover, limited by the requirement of a specific context, traditional therapy would cause poor air circulation in the space and the feeling of discomfort for the users.

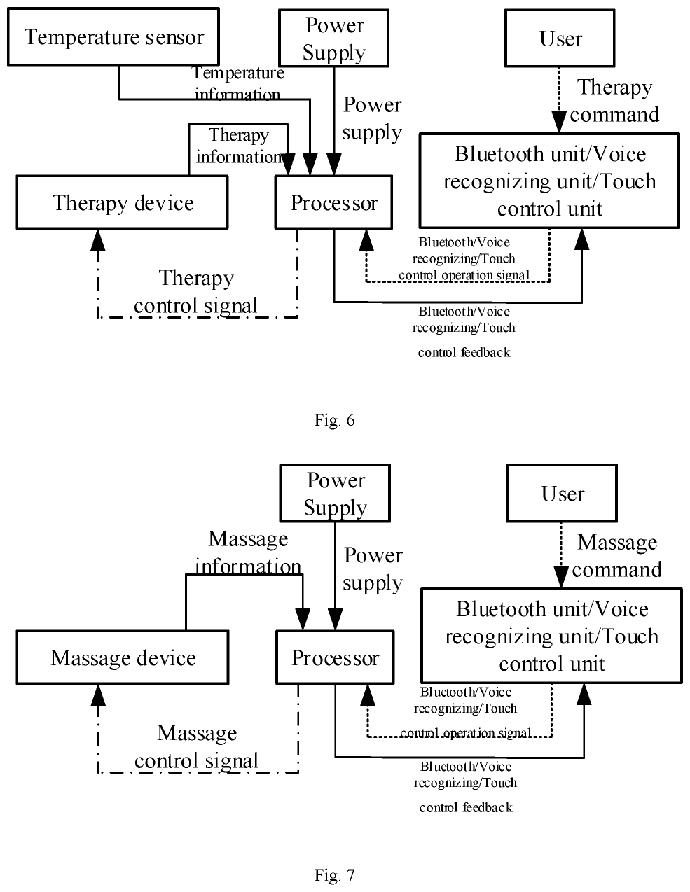

In order to solve these problems, in the embodiment of the present application, the therapy command issued by the user can be received by the Bluetooth unit, the voice recognizing unit, or the touch control unit to control the processor to regulate the operation of the therapy device. The therapy device includes a heater and a therapy bag. According to the principle of thermotherapy, when a user is sitting on the therapy device, the therapy device will heat the user's skin to increase the temperature of the body surface and accelerate the activity of the skin cells, so the capillaries on the surface of the skin dilate, and the impurities accumulated in the skin folds are excreted along with the sweat, thereby achieving the therapeutic effects of dredging the meridians, improving blood circulation, detoxifying, and beautifying. In this case, the pores all over the body have been stretched, and the skin will absorb all kinds of therapeutic nutrients inside the therapy bag of the therapy device. In this way, people can get treatment for the body parts simply by sitting or leaning on the smart cushion. As shown in FIG. 6, the Bluetooth command from the user can be received by the Bluetooth unit, the touch control command from the user can be received by the touch control unit, or the voice command from the user can be received by the voice recognizing unit. Further, the therapy command is converted into a therapy operation signal to be transmitted to the processor. It should be noted that here the processor is only allowed to receive one type of therapy operation signal at one time. The processor receives the therapy operation signal and sends a therapy control signal to regulate the operation of the therapy device. The smart cushion can be used as an independent therapy cushion product. In specific use, the Bluetooth unit, the voice recognizing unit, or the touch control unit receives the therapy command issued by the user and converts the therapy command into a therapy operation signal. The processor receives the therapy operation signal and sends a therapy control signal to the therapy device. The heater in the therapy device starts to heat the therapy bag in response to the therapy control signal received and gives feedback on the therapy information in real time. Moreover, the temperature sensor detects the temperature in the therapy cushion in real time and gives feedback to the user through the processor for the user's reference.

In addition, in the embodiment of the present application, the massage command issued by the user can be received by the Bluetooth unit, the voice recognizing unit, or the touch control unit to control the processor to regulate the operation of the massage device. With the quickened pace of people's lives and the increasing work pressure, the massage is a good way to stretch oneself physically and mentally. The massage chairs available now have complicated structures and high costs, and are bulky and cumbersome. Moreover, the massage chair mainly includes a chair body a massage stick configured inside the chair body. The massage stick realizes the massage purpose by rotating and moving back and forth against the human body. The massage chair has a structure of an ordinary recliner. The user lies on the massage chair for massage, and a certain part of the body gets massaged with squeezing actions to achieve the effects of stretching the body and relaxing the muscles and bones. On the downside, since the massage chair has default settings on the control modes, the more humanized needs and experiences of the users cannot be satisfied. The massage chair only having the simple and single massage function has no longer been able to meet the actual needs of users. Also, the massage chair having the single and manual operation and control mode cannot provide the user with more experiences. Limited by the use range, the large size, and the bulky property, the massage chair is not portable, and based on the factor that the massage chair can only be supplied with 220V electricity, the use of the massage chair is limited to specific occasions. Therefore, the user's needs of using the apparatus in multiple occasions at anytime and anywhere cannot be satisfied, and it is far from meeting user's real personalized demands and requirements.

In order to solve these problems, in the embodiment of the present application, the massage command issued by the user can be received by the Bluetooth unit, the voice recognizing unit, or the touch control unit to control the processor to regulate the operation of the massage device. The massage device includes a motor and a massage stick. The mechanical rolling actions and mechanical squeezing actions are applied for massage, so as to dredge the meridians, improve the blood circulation, and maintain the Yin-Yang balance of the body. After getting the massage, the users can feel that their muscles are relaxed, their joints are flexible, their spirit is refreshed, so the massage can eliminate the fatigue and play an important role in keeping healthy. People can enjoy the massage to the body parts simply by sitting or leaning on the smart cushion. As shown in FIG. 7, the Bluetooth command from the user can be received by the Bluetooth unit, the touch control command from the user can be received by the touch control unit, or the voice command from the user can be received by the voice recognizing unit. The massage command is converted into a massage operation signal to be transmitted to the processor. It should be noted that here the processor is only allowed to receive one type of massage operation signal at one time. The processor receives the massage operation signal and sends a massage control signal to control the operation of the massage device. The smart cushion can be used as an independent massage cushion product. In specific use, the Bluetooth unit, the voice recognizing unit, or the touch control unit receives the massage command issued by the user and converts the massage command into a massage operation signal. The processor receives the massage operation signal and sends a massage control signal to the massage device. The massage device gives feedback on massage information in real time for the user's reference in response to the massage control signal received.

In a specific embodiment of the present invention, the fan is a scroll-type exhaust fan.

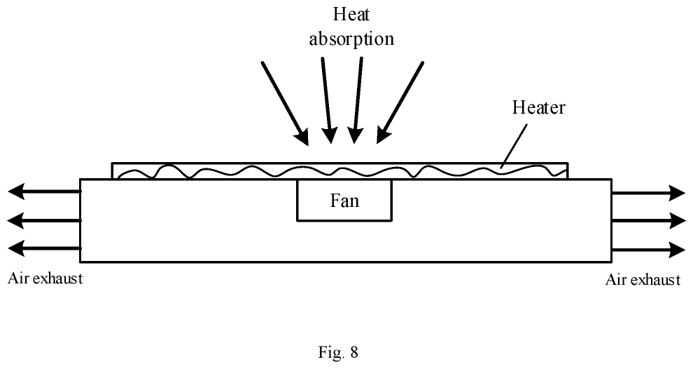

Specifically, most of the existing smart cushions with ventilation purpose use blowers. According to the blowing principle of the blower, the heat released by the human body is taken away by air extraction. However, due to the weight of the human body, the outflow air driven by the blower is blocked by the gravitational force of the human body, while the air intake is also blocked. As a result, the heat released by the human body cannot circulate, and thus the heat dissipation problem cannot be fundamentally solved. In order to solve this problem, in the embodiment of the present application, a scroll-type exhaust fan is used. As shown in FIG. 8, according to its air exhausting principle, the heater is located above the scroll-type exhaust fan for sucking the human body's heat into the main body of the smart cushion through the air inlet of the fan, and then discharging the human body's heat out of the smart cushion through the reflux guiding channel, so the problem of heat dissipation can be fundamentally solved.

In a specific embodiment of the present invention, the heater includes at least two layers of breathable materials and a heating wire arranged between two adjacent layers of breathable materials.

Specifically, most of the existing smart cushions with heating purpose use the enclosed heater materials, in which the upper and lower layers of fiber cloth materials enclose and seal the heating wire in the middle. When the heating wire is energized and heated, the heat generated by the heating wire can be held inside to achieve the heating purpose because the upper and lower layers of the fiber cloth materials are not breathable. However, since the human body can release a certain amount of heat, when a user is sitting on the smart cushion equipped with the enclosed heater, the heat from the two sources gets accumulated together, and the excess heat cannot be dissipated. Thus, the heat cannot circulate, and the case where the heat is accumulated locally can be caused, thereby providing a feeling of discomfort. In order to solve this problem, in the embodiment of the present application, a breathable heater is used, which includes at least two layers of breathable materials and a heating wire arranged between two adjacent layers of breathable materials. According to the principle of heat circulation, since the upper and lower layers of materials and the heating wire are breathable, when a user is sitting on the smart cushion, according to the ventilation principle of the smart cushion, the excess heat released by the human body can be dissipated, and the air flows around the gaps of the breathable materials. Based on the fact that the heat from the heater and the heat of the human body both can flow along with the air circulation, the heat circulation is improved. Therefore, the sultry and the feeling of discomfort caused by the local heat accumulation is fundamentally solved.

In a specific embodiment of the present invention, the reflux guiding channel includes at least two ventilation pipes perpendicular to each other; and

the fan is fixedly provided at an intersection of the two ventilation pipes.

Specifically, in order to better discharge the air sucked by the fan to reduce the noise caused by the air reflux of the fan, the embodiment of the present application provides at least two ventilation pipes perpendicular to each other inside the main body of the smart cushion. The ventilation pipes are designed to better solve the problems of heat reflux and noise during the operation of the fan. The excess heat and airflow can be directly exhausted out of the main body of the smart cushion through the ventilation pipes, and the noise that occurs during the operation of the fan can also be effectively reduced. When the fan is running, problems of noise and heat reflux will present. In order to reduce noise and heat reflux problems, the materials having sound insulation and ventilation effects are adopted in the structure design of the ventilation pipes, and the heat is discharged through the air guiding channel, so the noise is reduced. Therefore, the problem occurring in the discharging of air and heat can be fundamentally solved, and the noise problem occurring during the operation of the fan is alleviated to a greater extent.

In a specific embodiment of the present invention, a vacuum fixation installation system is further provided at the bottom of the main body of the smart cushion.

Specifically, the existing smart cushions are mostly fixed by sewing straps. Namely, the smart cushions are fixed on the chairs and other products by straps. However, the fixing method of using straps has a great limitation. For example, for a sofa or an office-specific boss chair, the smart cushion cannot be fixed by straps, so the smart cushion would slide and cannot be used normally, thereby resulting in potential safety risk. For some situations where the straps are applicable, for example, in terms of a car chair, the smart cushion should be fixed against the leather on the outer surface of the chair, wear trace will be caused on the smart cushion. In order to solve this problem, in the embodiment of the present application, a vacuum fixation installation system combined with a mechanical strap fixation is used. The vacuum fixation installation system is designed to achieve fixation and connection with the product to be connected by providing multiple vacuum suction devices at the bottom of the smart cushion. These vacuum suction devices are configured to remove the air from all areas coming in contact with the product to be connected. For example, in terms of the leather boss chairs, leather car chairs, and leather home sofas, the method for using the smart cushion is very simple, one just needs to put the smart cushion product on the leather product, and the suction devices at the bottom of the smart cushion will be attached to the surface material of the chair by suction, thereby achieving the fixation and satisfying the product requirements. In this way, the problem of difficulty in fixing the smart cushion on some products by the straps can be solved, while the damage to the smart cushion can be avoided, and the appearance is more attractive. Alternatively, the smart cushion of the present invention can also be fixed with straps. With the multi-purpose fixing method, the smart cushion not only can be fixed on the leather products such as office sofas, boss chairs, car chairs, home sofas, and some many by suction fixation, but also can be fixed on the office chairs, car chairs, and the like by the straps, suction fixation, or other fixing methods.

In addition, almost all the existing smart cushion products are in a fixed mode. Namely, all the accessories are fixed and sewn as a whole, which makes it large in size, bulky, inconvenient for cleaning, and can only be used on specific occasions. In either case, the economic aspect or the value embodiment of the product itself, the smart cushion cannot play a greater role. Therefore, in the embodiment of the present application, a smart cushion which can be combined in a simple way and is portable and applicable on any occasion is provided in consideration of the economic efficiency. The main body of the smart cushion includes a smart cushion cover, a structural portion, and a core portion. The smart cushion cover enables the smart cushion to have a more attractive appearance, more clean, and more convenient for cleaning. That is, when cleaning is desired, one only needs to take off the smart cushion cover and wash the cover directly, which is very convenient. The core portion is provided with the fan, the heater, the temperature sensor, the reflux guiding channel, the therapy device, and the massage device. The power supply and the controller are located in the structural portion, so the smart cushion product can have a relatively larger size and the users with larger body shapes can use it. Different people have different body shapes, either large or small, in order to allow more people with different body shapes to use the smart cushion, the structural portion is designed on purpose.

When a user with a large body shape uses the smart cushion, the core portion, i.e., the main portion of the smart cushion, can be installed in the structural portion. The structural portion is designed with an installation space for the main portion of the smart cushion. It is easy to use the smart cushion, i.e. one simply needs to put the core portion into a core portion recess of the structural portion. The design of the installation space for accommodating the core portion in the structural portion is to facilitate the installation and removal of the core portion.

When a user or a child with a small body shape uses the smart cushion, such a large size is not necessary. That is, when the structural portion is not required, the core portion can be simply taken out and used separately. The core portion is also an independent product. When traveling or on a business trip, the user can carry the core portion directly. Due to the light weight of the core portion, it is very convenient to carry around the core portion.

According to the multifunctional smart cushion of the present invention, a scroll-type exhaust fan is used to dissipate the heat in the smart cushion by means of air extraction, which avoids the problem of difficulty in timely discharging the heat from the parts blocked by the human body with the traditional blowing method. The air can be exhausted out of the smart cushion in time through the reflux guiding channel, which reduces heat reflux and noise. By providing the heater made of breathable material, the heat circulation inside the smart cushion is realized, which avoids the problem of local heat accumulation caused by poor heat circulation in the traditional heater. The vacuum fixation installation system is provided for suction fixation, which makes the smart cushion have a more attractive appearance and less likely to damage the smart cushion. In addition, the multifunctional smart cushion of the present invention has the functions of aroma diffusion, therapy, and massage, so it has various functions and wide application range. The multifunctional smart cushion can be used in various occasions such as office, home, car, train, airplane, cinema, waiting room, outdoor, field, travel, restaurant, etc.

The present invention is further described in detail above in combination with the specific preferred embodiments, and the specific embodiments of the present invention are not limited to the description. For those of ordinary skill in the art, various simple deductions or substitutions may be made without departing from the idea of the present invention, and these deductions or substitutions shall be considered as falling within the protection scope of the present invention.

What is claimed is:

1. A multifunctional smart cushion, comprising: a smart cushion main body and a mobile terminal, wherein, the smart cushion main body comprises a smart cushion cover, a structural portion, and a core portion; the structural portion is located around the core portion; and the smart cushion cover covers the core portion and the structural portion;
the structural portion is provided with a power supply and a controller;
the core portion is provided with a fan, a heater, a temperature sensor, a reflux guiding channel, a therapy device, and a massage device;
the fan, the heater, the temperature sensor, the therapy device, and the massage device are all connected to the power supply through the controller; and
the mobile terminal is connected to the controller via a wireless communication.

2. The multifunctional smart cushion according to claim 1, wherein, the power supply is a mobile power supply, and a voltage of the mobile power supply does not exceed a safety voltage level for the human body.

3. The multifunctional smart cushion according to claim 1, wherein, the controller comprises:
a processor, configured to receive an operation signal, and send a control signal;
a switch button, configured to switch between different working modes;
a Bluetooth unit, configured to receive a Bluetooth command sent by a user, and convert the Bluetooth command into a Bluetooth operation signal;
a voice recognizing unit, configured to receive a voice command issued by the user, and convert the voice command into a voice operation signal; and
a touch control unit, configured to receive a touch control command issued by a user, and convert the touch control command into a touch control operation signal; wherein
the Bluetooth unit, the voice recognizing unit, and the touch control unit are all connected to the processor through the switch button.

4. The multifunctional smart cushion according to claim 3, wherein, the Bluetooth command comprises a Bluetooth operation command or a Bluetooth voice command.

5. The multifunctional smart cushion according to claim 1, wherein, the controller further comprises:
an aroma diffusing unit, connected to the processor, configured to act in response to an aroma diffusing control signal sent by the processor.

6. The multifunctional smart cushion according to claim 1, wherein, the fan is a scroll-type exhaust fan.

7. The multifunctional smart cushion according to claim 1, wherein, the heater comprises at least two layers of breathable materials and a heating wire arranged between two adjacent layers of breathable materials.

8. The multifunctional smart cushion according to claim 1, wherein, the reflux guiding channel comprises at least two ventilation pipes perpendicular to each other; and
the fan is fixedly provided at an intersection of the two ventilation pipes.

9. The multifunctional smart cushion according to claim 1 further comprising a vacuum fixation installation system provided at a bottom of the smart cushion main body.

10. The multifunctional smart cushion according to claim 2 further comprising a vacuum fixation installation system provided at a bottom of the smart cushion main body.

11. The multifunctional smart cushion according to claim 3 further comprising a vacuum fixation installation system provided at a bottom of the smart cushion main body.

12. The multifunctional smart cushion according to claim 4 further comprising a vacuum fixation installation system provided at a bottom of the smart cushion main body.

13. The multifunctional smart cushion according to claim 5 further comprising a vacuum fixation installation system provided at a bottom of the smart cushion main body.

14. The multifunctional smart cushion according to claim 6 further comprising a vacuum fixation installation system provided at a bottom of the smart cushion main body.

15. The multifunctional smart cushion according to claim 7 further comprising a vacuum fixation installation system provided at a bottom of the smart cushion main body.

16. The multifunctional smart cushion according to claim 8 further comprising a vacuum fixation installation system provided at a bottom of the smart cushion main body.
